SEQUENCE LISTING (4 sequences)

SEQ ID NO: 1 - DNA, 1041 bp, Pseudomonas aeruginosa
  source 1..1041: mol_type = other DNA
atgcgtcacggggatatttcgtcgtcgaatgacacggtcggggtcgctgtcgtcaactataagatgcctcgcttgcacacggccgctgaagtgctcgataacgcccgtaagattgcagagatgatcgtcgggatgaaacaggggctgcctgggatggacttggtcgtctttccggagtactcgctgcaaggcattatgtatgatccagccgaaatgatggaaacggctgtggctatccctggcgaagaaaccgaaatcttctcgcgtgcctgtcgcaaagcgaacgtctggggtgtgttttccctcaccggcgagcgtcacgaagaacatccccggaaggccccctataacacgctggtcctcatcgacaacaacggtgaaattgtccaaaagtatcgtaagatcatcccctggtgtccgatcgaagggtggtatccgggcggtcagacctacgtgagcgaagggccaaaagggatgaaaattagcctcattatttgcgacgatggcaactaccctgaaatctggcgcgactgcgcaatgaagggcgctgagttgattgtccggtgtcagggctatatgtaccccgctaaggaccaacaggtcatgatggctaaagcaatggcctgggcaaacaactgctatgtggcagtggcgaacgcagcaggtttcgatggggtctactcctattttggccactcggccatcatcgggttcgacgggcgtacgttgggcgagtgcggcgaagaagaaatgggtattcagtatgcgcagttgtccctgtcgcaaatccgtgatgctcgggcgaatgaccaaagccaaaaccacctgtttaagattctccatcgcgggtatagcggtctccaggcctccggcgacggcgaccgcggtctggctgaatgcccctttgagttttaccgcacgtgggtgacggacgccgaaaaggcgcgtgaaaacgtggagcgcttgacccgtagcaccaccggtgtggcacagtgcccagtcggtcggctcccctatgaagggttggagaaagaggcttga

SEQ ID NO: 2 - protein, 346 aa, Pseudomonas aeruginosa [sequence omitted]

SEQ ID NO: 3 - DNA, 1410 bp, Pseudomonas putida
  source 1..1410: mol_type = other DNA
atgacccagaaaaccgcgattggcgaactcaccgccgtcgaactgctcgaactgtaccatcgccgccagttgtcacccgtcgaggtggtcgacgatgtgctggcgcgtatcgacctgcataacccggcggtcaacgccttctgccatgtcgacggcgaaggcgcccgcgccgccgcccgtgccagcgaacagcgctggcaacgtggccaaccctgtggccgactggatggcgtacccgcctcgatcaaggacctgaccctgacccgcggcatgcctacccgcaagggctcgcgtactacctcgggtgccgggccgtgggaaatcgatgcgcccttcagcgccttcatgcgtgaagccggtgcagtgctggtgggcaagaccaccacgccagagttcggctggaaaggggtgaccgacaacccgctgtacggcatcactcgcaacccgtgggacaccaggctgactgccggtggttcgtctggtggtgctgcggcagccgccgcgctgaacctgggggtgctgcatcagggcagcgatgccggtggttcgattcgcatcccttgcgccttcaccggcaccttcggcatcaaaccgaccttcggctatgtgccgcaatggccggccagcgccatgaccgtgctgtcgcacctggggccgatgacgcgtaccgtggacgactcggtgctgatgctcgactgcgtagcgcgccccgatgcccgcgacggtttggccggcgcaccccggcaggcgccgtggctcagccagcagcaagatctgagcggcctacgcatcgcctacagcgccaacttcggttacgtgcaggtcgacccacagatccaggcactggtggcgcaagcggtgcagcgcctggcgcggctgggtgcgcaggtagaggaagtggacccgggcttcagcgacccgctggaaaccttcaacaccctgtggtttgccggcgcggcgcggttggccagtgccttgagcgatgagcagaaagcgttgcttgacccggggctgcgctggattgccgaacaaggtgcacagatcagccttggcgaatacacccaggcgcttgaggcacgggcagaattgattgcgaagatgaatgcctttcatcagcgatatgacgtgctggtatcgccgatgctgccgttggtggcgttcgaagccggacacaacgtgccgcctgggtcggggatggcgcaatggatggagtggacgccgttaagctatccgttcaacctgacccaacagccggcggcctcggtaccctgcgggttcacccgcgaggggttgccggtgggcctgcaggtggtggccgggcgctttgctgatgagcaagtgctgcgggtgtgcaaggtgtatgagcagcattatccgagccggcacctgcaagcaccgattactggctga

SEQ ID NO: 4 - protein, 469 aa, Pseudomonas putida
MTQKTAIGELTAVELLELYHRRQLSPVEVVDDVLARIDLHNPAVNAFCHVDGEGARAAARASEQRWQRGQPCGRLDGVPASIKDLTLTRGMPTRKGSRTTSGAGPWEIDAPFSAFMREAGAVLVGKTTTPEFGWKGVTDNPLYGITRNPWDTRLTAGGSSGGAAAAAALNLGVLHQGSDAGGSIRIPCAFTGTFGIKPTFGYVPQWPASAMTVLSHLGPMTRTVDDSVLMLDCVARPDARDGLAGAPRQAPWLSQQQDLSGLRIAYSANFGYVQVDPQIQALVAQAVQRLARLGAQVEEVDPGFSDPLETFNTLWFAGAARLASALSDEQKALLDPGLRWIAEQGAQISLGEYTQALEARAELIAKMNAFHQRYDVLVSPMLPLVAFEAGHNVPPGSGMAQWMEWTPLSYPFNLTQQPAASVPCGFTREGLPVGLQVVAGRFADEQVLRVCKVYEQHYPSRHLQAPITG

(19) United States
(12) Patent Application Publication
(10) Pub. No.: US 2026/0062721 A1
(43) Pub. Date: Mar. 5, 2026

(54) ENZYMES FOR THE BIOLOGICAL CATABOLISM OF ACETAMIDE FROM OXIDIZED NYLON WASTE

(71) Applicant: Alliance for Sustainable Energy, LLC, Golden, CO (US)

(72) Inventors: Ross EAGLESFIELD, Denver, CO (US); Allison Jean ZIMONT WERNER, Denver, CO (US); Gregg Tyler BECKHAM, Golden, CO (US)

(21) Appl. No.: 19/289,005

(22) Filed: Aug. 2, 2025

Related U.S. Application Data
(60) Provisional application No. 63/678,665, filed on Aug. 2, 2024.

(51) Int. Cl. C12P 7/40; C12N 9/80; C12N 15/70

(52) U.S. Cl. CPC C12P 7/40; C12N 9/80; C12N 15/70; C12Y 305/01004; C12N 2800/101

(57) ABSTRACT

The present disclosure relates to an engineered microorganism having an exogenous gene encoding an amidase, where the microorganism is capable of catabolizing acetamide to produce a carboxylic acid without the use of an additional substrate.

### CROSS-REFERENCE TO RELATED APPLICATIONS

This application claims priority from U.S. Provisional Patent Application No. 63/678,665 filed on Aug. 2, 2024 and its associated appendix, the contents of which are incorporated herein by reference in their entirety.

### CONTRACTUAL ORIGIN

This invention was made with government support under Contract No. DE-AC36-08GO28308 awarded by the Department of Energy. The government has certain rights in the invention.

### SEQUENCE LISTING

The instant application contains a Sequence Listing which has been submitted electronically and is hereby incorporated by reference in its entirety. The XML copy as filed herewith was originally created on Jul. 23, 2025 is named “NREL 24-108.xml” and is 7,700 bytes in size.

### BACKGROUND

Fibrous polymeric materials such as poly (ethylene terephthalate) (PET) and polyamides, or nylons, have revolutionized many industries, especially in the textiles industry. The desirable mechanical properties and inexpensive synthesis of these fibers has facilitated a shift from natural polymers such as cotton to these petrochemically-derived alternatives. This has provided companies with a large benefit in terms of economics. Global production of PET fibers is around 50 million metric tons, a significant proportion of total polyester synthesis, with the majority being used by the textile industry. Nylon-6 and nylon-6,6 are the most common derivations of nylon and are produced at a scale of around 5-6 million metric tons per year mainly for use in textiles and fishing nets. Forecasts anticipate that these production values will only increase in the future.

Recycling these materials has remained a significant challenge and must be addressed to achieve a circular carbon economy non-reliant on fossil fuels. Mixed plastic streams, i.e. textiles, are particularly difficult to efficiently recycle due to a mismatch in reactivity between polyesters, which typically require harsher reaction conditions, and nylons which can be easily overoxidized. As a result, it is currently easier and more economical to synthesize these materials from virgin feedstocks than it is to recycle or re-use them. Moreover, while PET is currently one of the most well recycled consumer plastics, nylons are rarely recycled with most post-consumer materials going to landfill or being incinerated. Incineration of nylons can lead to the release of harmful gaseous byproducts such as hydrogen cyanide and nitrous oxide, while landfilled material will slowly break down into microplastics which contaminate many environments. Nylon waste is also pervasive and recalcitrant in the world's oceans. Most of the plastic waste generated by the fishing industry contains nylon and contributes heavily to the microplastics catastrophe being played out in the oceans.

One chemical depolymerization strategy leverages the oxidative breakdown of C—C bonds to depolymerize into small molecule oxygenates. The operative mechanism is believed to involve the generation of alkyl radicals at C—H bonds on a polymer backbone, followed by radical coupling with O₂ and the formation of peroxide intermediates. These intermediates are then homolytically cleaved at the O—O bond in the presence of Co and Mn catalysts forming alkoxyl radicals, thus triggering β-scission of C—C bonds on the polymer backbone and generating oxygenated species.

One such oxygenated species resulting from the oxidative deconstruction of nylon-6 is acetamide, in addition to various nitrogenated compounds and short chain (C₄—C₆) dicarboxylic acids (DCAs). Thus, there remains a need for systems, methods, and compositions that can metabolize and/or catabolize acetamide.

### SUMMARY

An aspect of the present disclosure is an engineered microorganism having an exogenous gene encoding an amidase, where the microorganism is capable of catabolizing acetamide to produce a carboxylic acid without the use of an additional substrate. In some embodiments of the present disclosure, the additional substrate may include a sugar, an alcohol, a protein, and/or a nucleic acid. In some embodiments of the present disclosure, the carboxylic acid is acetate.

In some embodiments of the present disclosure, the engineered microorganism may be a species ofPseudomonas. In some embodiments of the present disclosure, the species may include a strain of Pseudomonas putida. In some embodiments of the present disclosure, the strain is a strain of KT2440. In some embodiments of the present disclosure, the exogenous gene may be derived from a species of Pseudomonas that is not Pseudomonas putida. In some embodiments of the present disclosure, the exogenous gene may be derived from Pseudomonas aeruginosa.

In some embodiments of the present disclosure, the exogenous gene may include amiE. In some embodiments of the present disclosure, the exogeneous gene may have a nucleic acid sequence that is at least 80% identical to SEQ ID NO:1. In some embodiments of the present disclosure, the amidase may have an amino acid sequence that is at least 80% identical to SEQ ID NO:2. In some embodiments of the present disclosure, the exogenous gene may be chromosomally incorporated into the engineered microorganism.

In some embodiments of the present disclosure, the engineered microorganism may further include the deletion of an endogenous gene encoding an amidase. In some embodiments of the present disclosure, the endogenous gene may have a nucleic acid sequence that is at least 80% identical to SEQ ID NO:3. In some embodiments of the present disclosure, the endogenous gene may encode an amino acid sequence that is at least 80% identical to SEQ ID NO:4.

An aspect of the present disclosure is a method that includes depolymerizing a polyamide to produce a mixture that includes acetamide and using an engineered microorganism, converting at least a portion of the acetamide to a carboxylic acid, where the engineered microorganism includes an exogenous gene encoding an amidase, and the microorganism is capable of catabolizing acetamide to produce a carboxylic acid without the use of an additional substrate.

### BRIEF DESCRIPTION OF DRAWINGS

Some embodiments are illustrated in referenced figures of the drawings. It is intended that the embodiments and figures disclosed herein are to be considered illustrative rather than limiting.

FIG. 1 illustrates a microtiter plate growth assay using plasmid-based overexpression strains of P. putida KT2440 expressing native amidases using the pBTL-2 overexpression vector, according to some embodiments of the present disclosure. Media was M9 bacterial media supplemented with 50 mM acetamide. pAW074-PP_0613 overexpression; pAW075-PP_2932overexpression; pA W076-PP_5629 overexpression. All data are mean±SD from 3 independent replicates.

FIG. 2 illustrates a microtiter plate growth assay using plasmid-based overexpression strain YC026 containing pAW074 containing the putative amidase PP_0613 (Panel A) compared to a non-inoculated control (Panel B), according to some embodiments of the present disclosure. Media was M9 minimal media supplemented with titrations of acetamide from 0-200 mM as the sole carbon source. All data are mean±SD from 3 independent replicates.

FIGS. 3A illustrates plasmid-based overexpression of amidase enzymes for acetamide utilization in Pseudomonas putida KT2440, according to some embodiments of the present disclosure, results from shake-flask experiments analyzing OD₆₀₀, glucose, acetamide, and acetic acid quantification in cells grown without (Panel A) or with 20 mM exogenous glucose (Panel B), respectively. All data are mean±SD from 3 independent replicates.

FIG. 3B illustrates plasmid-based overexpression of amidase enzymes for acetamide utilization in Pseudomonas putida KT2440, a schematic representation of P. putida strain YC039 overexpressing PP_0613, according to some embodiments of the present disclosure,

FIG. 3C illustrates results from shake-flask experiments using YC039 analyzing OD₆₀₀, glucose, acetamide, and acetic acid quantification in cells grown without (Panel A) or with 20 mM exogenous glucose (Panel B), respectively, according to some embodiments of the present invention. All data are mean±SD from 3 independent replicates.

FIG. 3D illustrates plasmid-based overexpression of amidase enzymes for acetamide utilization in Pseudomonas putida KT2440, a schematic representation of P. putida strain RE016 overexpressing amiE from Pseudomonas aeruginosa, according to some embodiments of the present disclosure.

FIG. 3E illustrates results from shake-flask experiments using RE016 analyzing OD₆₀₀, glucose, acetamide, and acetic acid quantification in cells grown without (Panel A) or with 20 mM exogenous glucose (Panel B), respectively, according to some embodiments of the present invention. All data are mean±SD from 3 independent replicates.

FIGS. 4A illustrates microtiter plate growth experiments of P. putida WT, according to some embodiments of the present disclosure. Panel A illustrates data collected for cells grown in the absence of glucose and Panel B illustrates data for cells grown with 20 mM glucose. Acetamide was added to the media in the given concentrations from 0-200 mM. All data are mean±SD from 3 independent replicates.

FIGS. 4B illustrates microtiter plate growth experiments of YC039, according to some embodiments of the present disclosure. Panel A illustrates data collected for cells grown in the absence of glucose and Panel B illustrates data for cells grown with 20 mM glucose. Acetamide was added to the media in the given concentrations from 0-200 mM. All data are mean±SD from 3 independent replicates.

FIGS. 4C illustrates microtiter plate growth experiments of RE016, according to some embodiments of the present disclosure. Panel A illustrates data collected for cells grown in the absence of glucose and Panel B illustrates data for cells grown with 20 mM glucose. Acetamide was added to the media in the given concentrations from 0-200 mM. All data are mean±SD from 3 independent replicates.

FIG. 5A illustrates a schematic representation showing genome integrated strain RE026 with P_{tac} promoter insertion into the PP_0613 locus overexpressing amidase enzymes, according to some embodiments of the present disclosure.

FIG. 5B illustrates growth and substrate utilization of genome integrated strain RE026 illustrated in FIG. 5A grown in media containing no additional nitrogen, microtiter plate growth experiments measuring OD₆₀₀ values at varying acetamide concentrations (0-200 mM) in the absence (Panel A) and presence (Panel B) of 20 mM glucose, according to some embodiments of the present disclosure. All data are mean±SD from 3 independent replicates.

FIG. 5C illustrates shake-flask experiments using genome integrated strain RE026 illustrated in FIG. 5A measuring OD₆₀₀, glucose, acetamide, and acetic acid concentrations without (Panel A) and with (Panel B) glucose feeding at 0 and 24 hours, according to some embodiments of the present disclosure. All data are mean±SD from 3 independent replicates.

FIG. 5D illustrates a schematic representation of genome integrated strain RE020 with P_{tac} amiE insertion replacing the PP_0613 gene, according to some embodiments of the present disclosure.

FIG. 5E illustrates growth and substrate utilization of genome integrated strain RE020 illustrated in FIG. 5D grown in media containing no additional nitrogen, microtiter plate growth experiments measuring OD₆₀₀ values at varying acetamide concentrations (0-200 mM) in the absence (Panel A) and presence (Panel B) of 20 mM glucose, according to some embodiments of the present disclosure. All data are mean±SD from 3 independent replicates.

FIG. 5F illustrates shake-flask experiments using genome integrated strain RE020 illustrated in FIG. 5D measuring OD₆₀₀, glucose, acetamide, and acetic acid concentrations without (Panel A) and with (Panel B) glucose feeding at 0 and 24 hours. All data are mean±SD from 3 independent replicates.

FIG. 6A illustrates growth of P. putida strains in microtiter plates in M9 minimal media containing 1 g/L NH₄Cl and the noted carbon source/s without glucose, according to some embodiments of the present disclosure. WT (Panel A), RE026 (Panel B), and RE020 (Panel C). All data are mean±SD from 3 independent replicates.

FIG. 6B illustrates growth of P. putida strains in microtiter plates in M9 minimal media containing 1 g/L NH₄Cl and the noted carbon source/s with 20 mM glucose, according to some embodiments of the present disclosure. WT (Panel A), RE026 (Panel B), and RE020 (Panel C). All data are mean±SD from 3 independent replicates.

FIG. 7A illustrates biological substrate utilization and BKA production by RE022 grown with mock substrates, according to some embodiments of the present disclosure. (Panel A) Amalgamated data showing growth, substrate utilization, and BKA production from triplicate shake-flask experiment. (Panel B) Consumption of the short chain dicarboxylic acids (DCAs) succinic, glutaric, and adipic acid. All data are mean±SD from 3 independent replicates.

FIG. 7B illustrates biological substrate utilization and BKA production by RE022 grown with mock substrates, according to some embodiments of the present disclosure. (Panel A) Terephthalate utilization and BKA production. (Panel B) Acetamide consumption and subsequent acetate production and consumption. (Panel C) OD₆₀₀ readings and supernatant pH measured following cell removal by centrifugation. All data are mean±SD from 3 independent replicates.

FIG. 8A illustrates an engineered terephthalate degradation pathway highlighting the knockout of pcaD for muconolactone accumulation in P. putida, according to some embodiments of the present disclosure.

FIG. 8B illustrates biological substrate utilization and muconolactone production by RE093 grown with mock substrates and oxidized commercial PET and nylon-6, according to some embodiments of the present disclosure, specifically the quantification of short chain dicarboxylic acid (DCA) consumption succinic, glutaric, and adipic acid: (i) mock, (ii) mock with 20 mM glucose, (iii) effluent, (iv) effluent with 20 mM glucose. All data are mean±SD from 3 independent replicates.

FIG. 8C illustrates biological substrate utilization and muconolactone production by RE093 grown with mock substrates and oxidized commercial PET and nylon-6, according to some embodiments of the present disclosure, specifically acetamide and acetate quantification: (i)-(iv) arranged as in FIG. 8B. All data are mean±SD from 3 independent replicates.

FIG. 8D illustrates biological substrate utilization and muconolactone production by RE093 grown with mock substrates and oxidized commercial PET and nylon-6, according to some embodiments of the present disclosure, specifically terephthalate utilization and muconolactone production: (i)-(iv) arranged as in FIG. 8B. All data are mean±SD from 3 independent replicates.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

The present disclosure relates to the use of amidase enzymes PP_0613 from Pseudomonas putida KT2440 and AmiE from Pseudomonas aeruginosa for the biological catabolism of acetamide from oxidized nylon waste. More specifically, the amidase enzymes PP_0613 and AmiE have been overexpressed in Pseudomonas putida KT2440, which, as a result, was capable of deaminating acetamide into ammonium and acetate. The overexpression of either of these genes allowed this reaction to occur and for the cells to use acetamide as the sole source of carbon and nitrogen for cell growth. Acetamide is a key molecule that is produced during the oxidative deconstruction of nylon-containing waste materials. As such it is a target for biological utilization to allow the valorization of nylon-containing mixed textile waste using engineered bacteria.

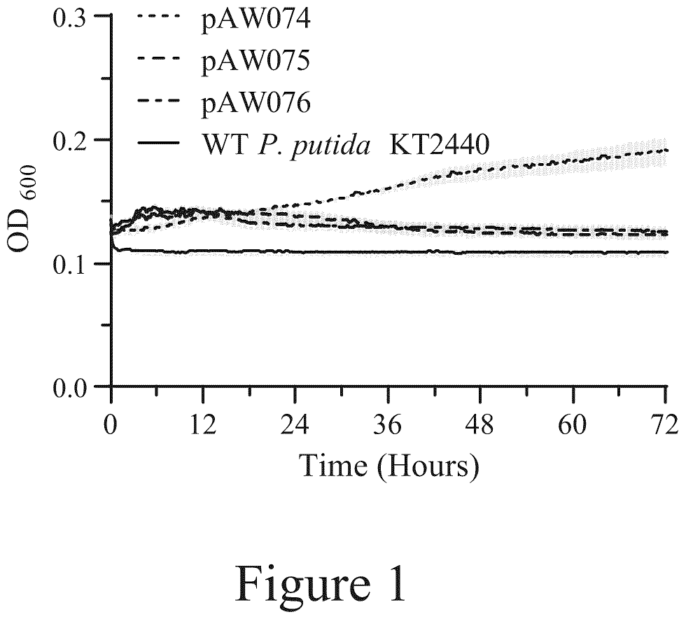

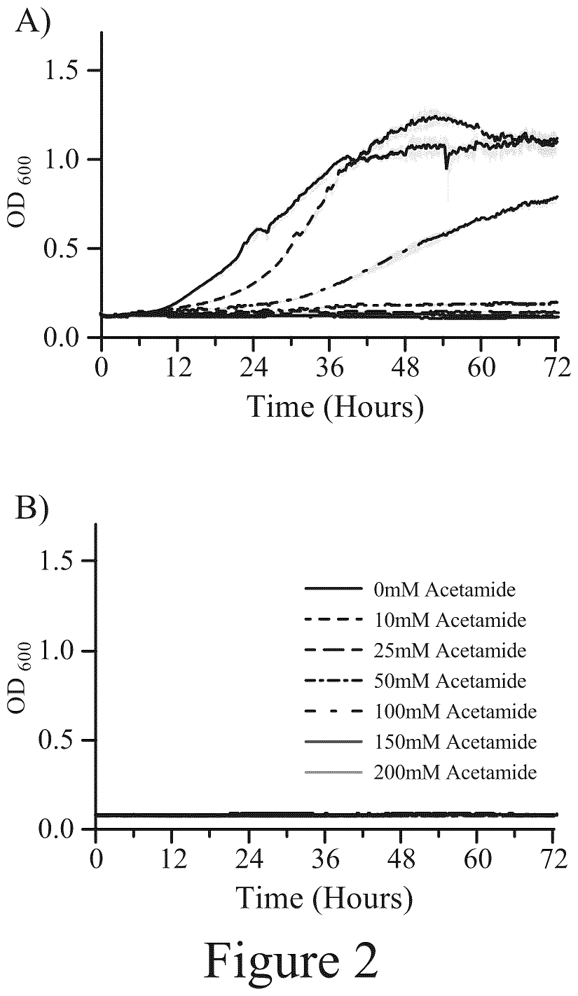

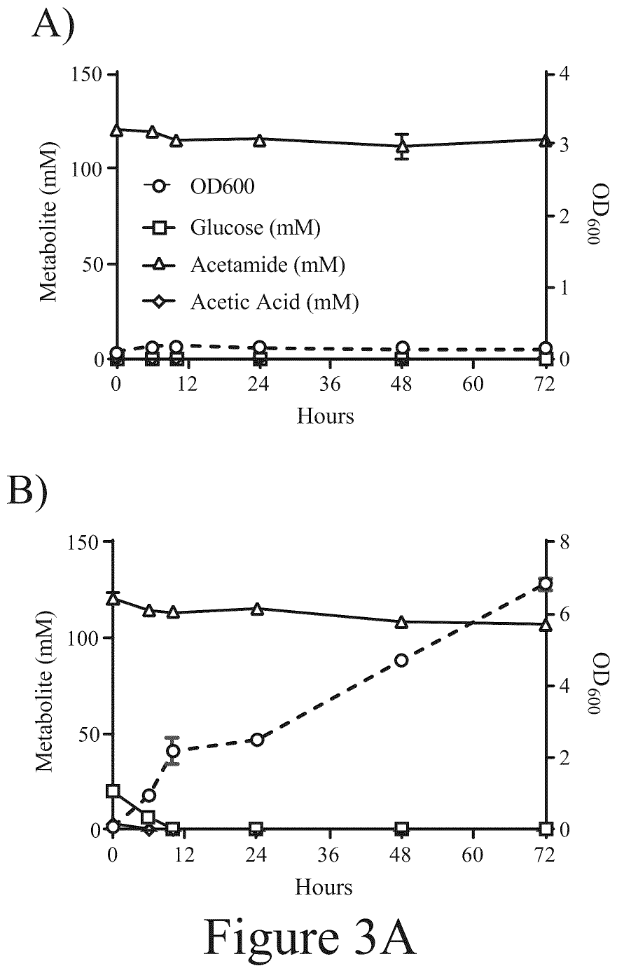

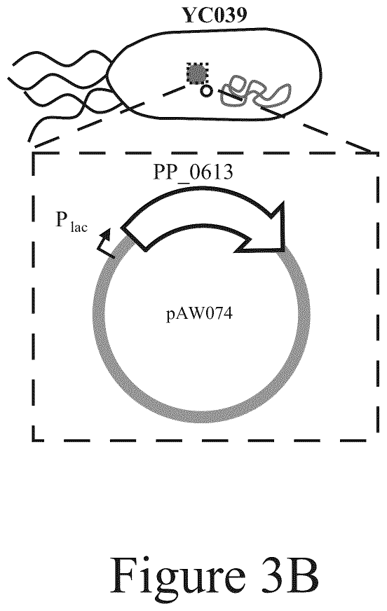

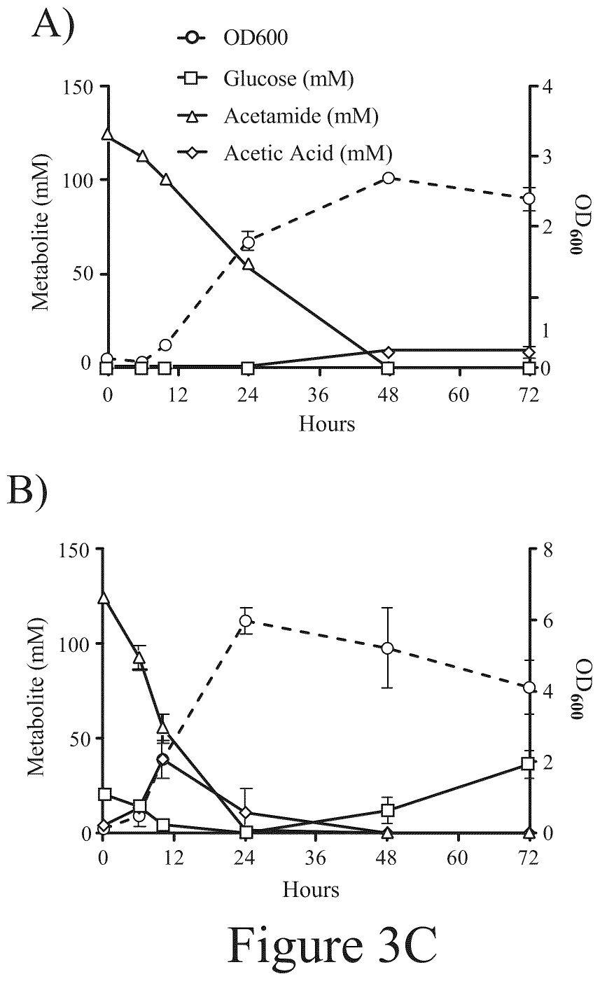

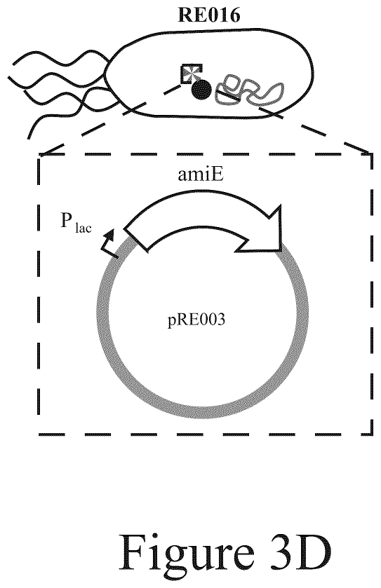

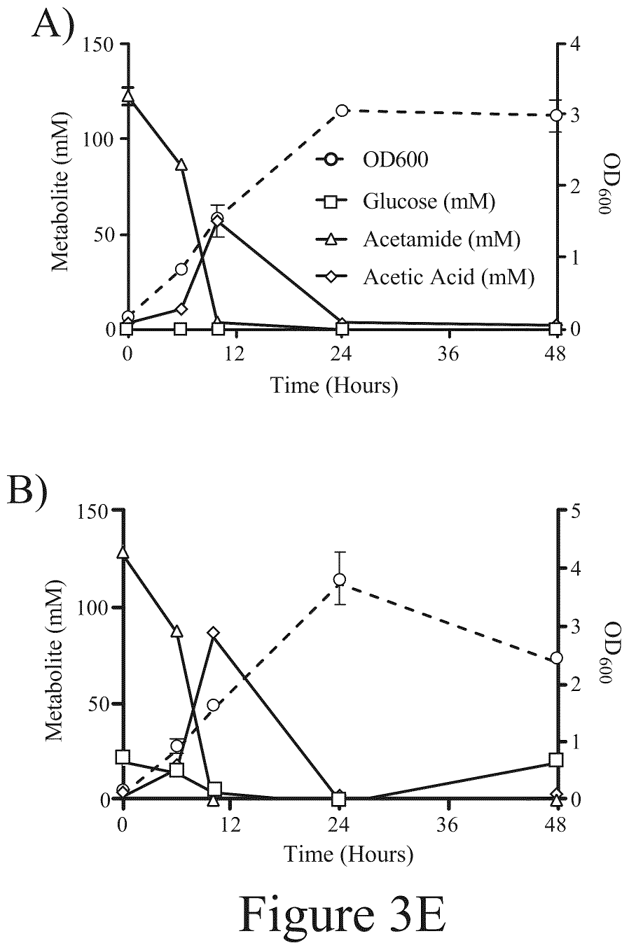

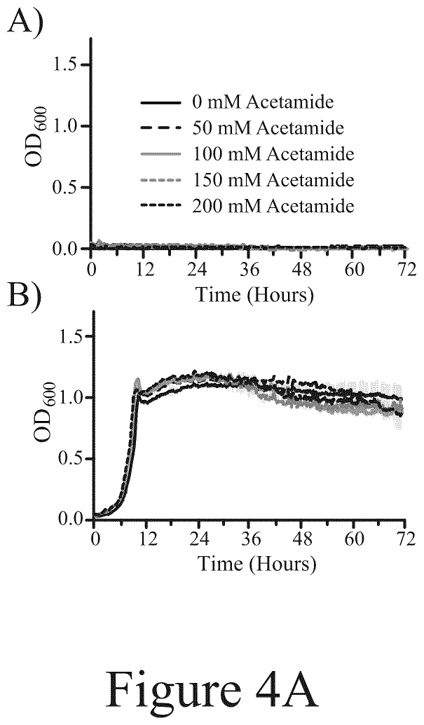

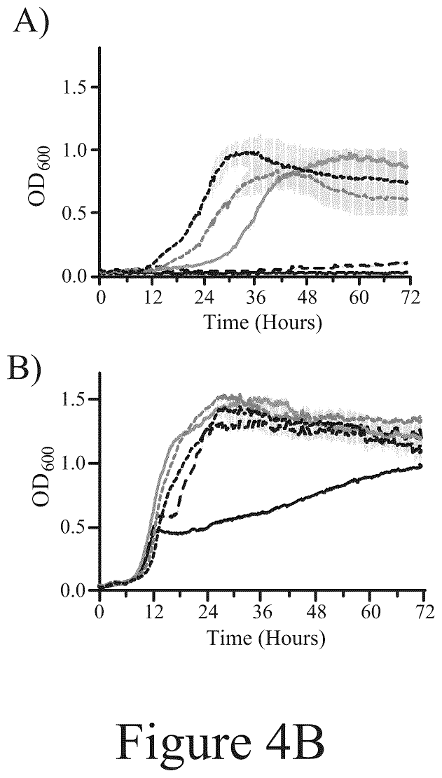

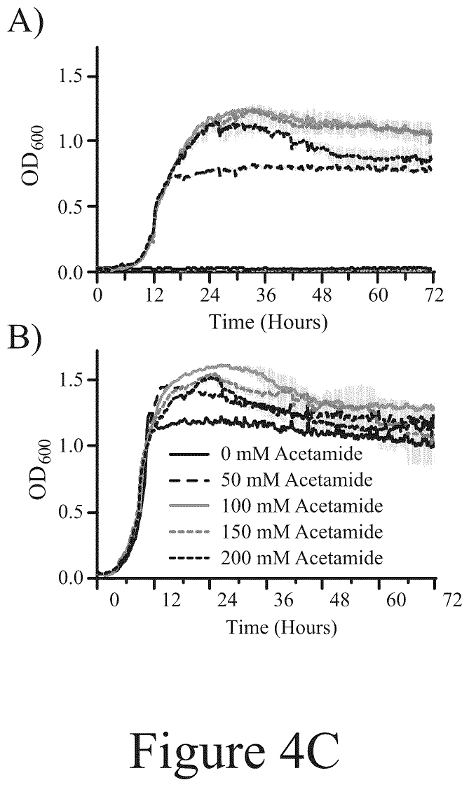

Pseudomonas putida can natively metabolize acetate to acetyl-CoA. However, it is unable to deaminate acetamide to acetate. Three enzymes within the KT2440 genome are annotated as amidases with the potential to perform this reaction: PP_0613, PP_2932, and PP_5629. Therefore, all three of these genes were cloned into a pBTL-2 overexpression vector under the control of the lac promoter and over-expressed in wild-type (WT) P. putida KT2440 (pAW074, 075, and 076 respectively). Only one, PP_0613 (strain YC039), was able to grow at all on 50 mM acetamide, albeit slowly (see FIG. 1). An acetamide titration showed reduced lag and increased growth rate at acetamide concentrations of up to 200 mM (see FIG. 2). With glucose as the sole carbon source growth of YC039 was unable to match WT, however, in the presence of acetamide and glucose OD₆₀₀ values exceeded those of the WT strain suggesting that acetamide was being metabolized to acetyl-CoA and used for growth (see FIGS. 3A-3E and FIGS. 4A-4C). HPLC analysis confirmed that acetamide was not utilized by the WT strain (see FIG. 3A) but was completely depleted by 48 hours in YC039 grown without glucose (see FIGS. 3B and 3C). With glucose, acetamide was utilized completely by 24 hours. However, the cells did not grow beyond this point, and additional fed glucose accumulated in the media (see Panel B of FIG. 3C).

While a native gene was initially targeted to perform the deamination reaction, it was decided to investigate heterologous expression of an amidase from a closely related Pseudomonad, AmiE from P. aeruginosa, which is known to be responsible for acetamide deamination in this bacterium. Overexpression of the amiE gene on pBTL-2 in WT P. putida KT2440 (strain RE016) resulted in reduced lag time and better growth on all acetamide concentrations when compared to YC039. Additionally, in the presence of glucose as the sole carbon source, the lag time was indistinguishable from WT (see FIG. 3E and FIGS. 4A-4C). Like YC039, the addition of acetamide alongside glucose led to increased levels of growth (see FIGS. 4A-4C). HPLC analysis confirmed that acetamide was utilized almost completely by 10 hours of growth and a large quantity of acetate was detected at 10 hours post inoculation in media both containing and lacking glucose (see FIG. 3E).

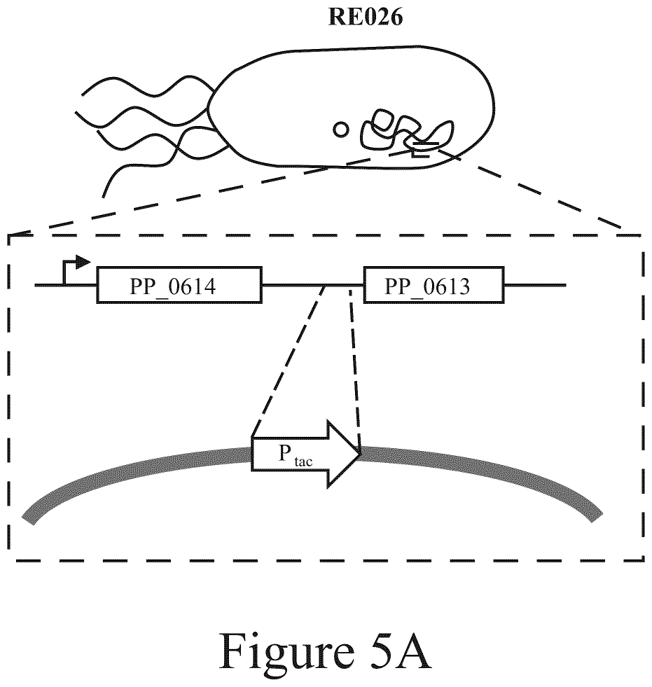

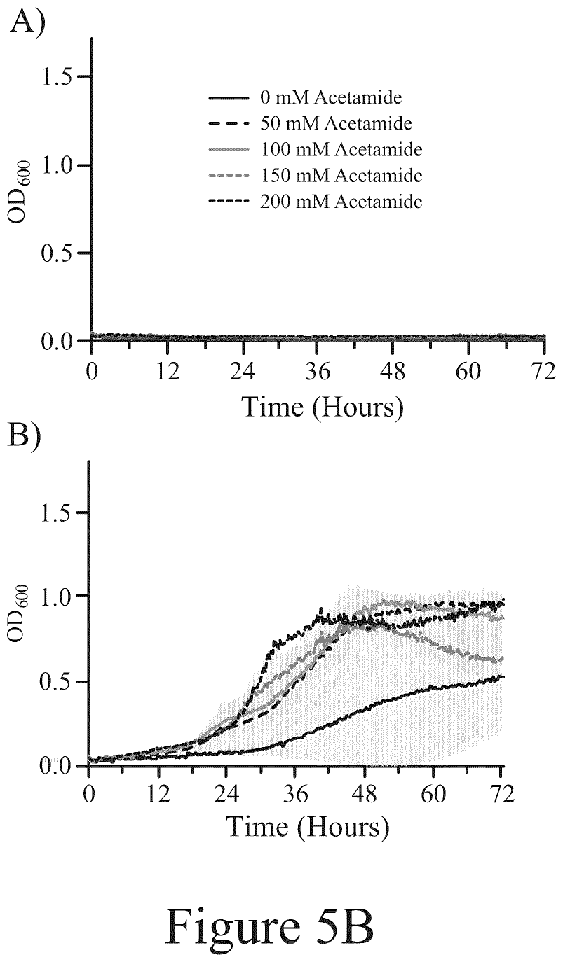

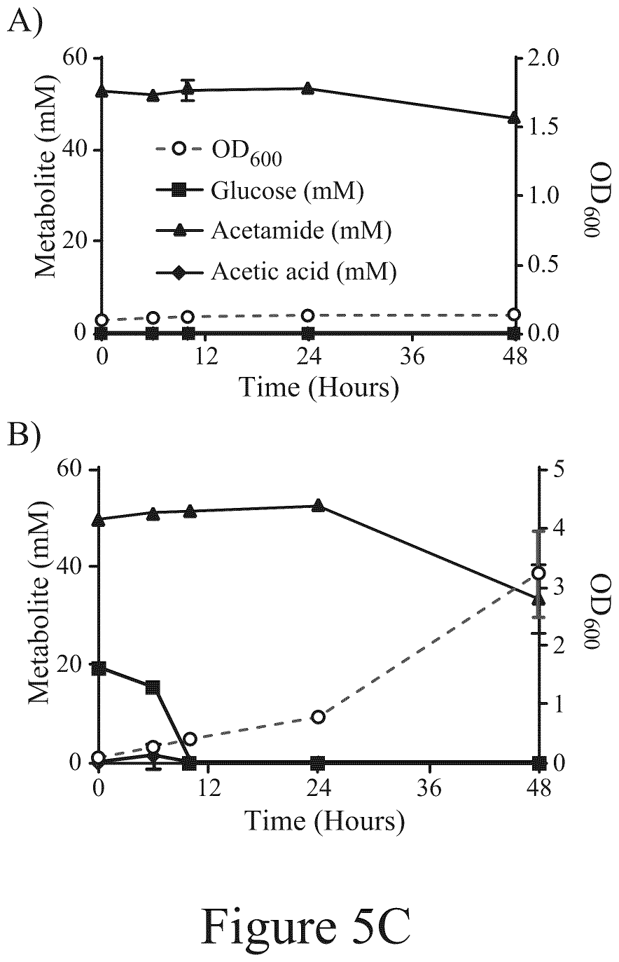

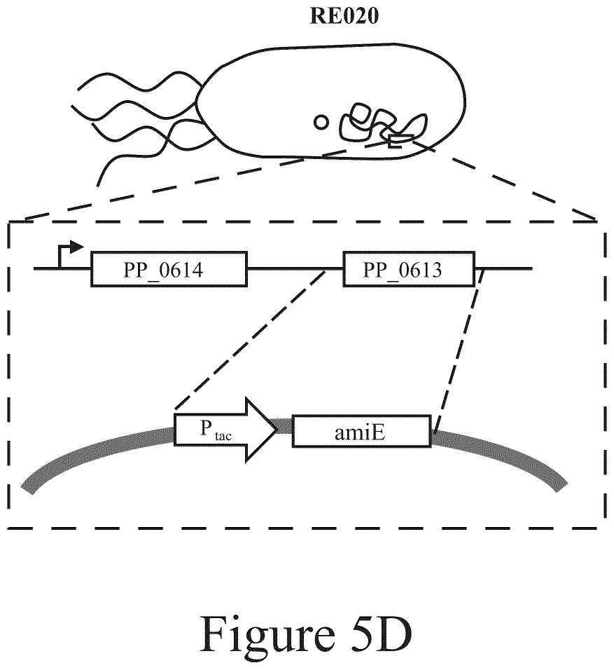

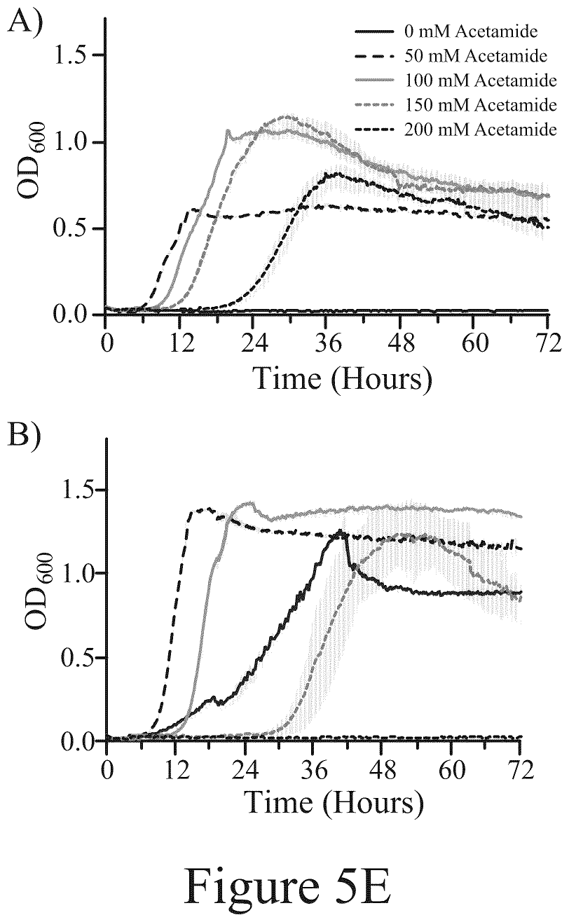

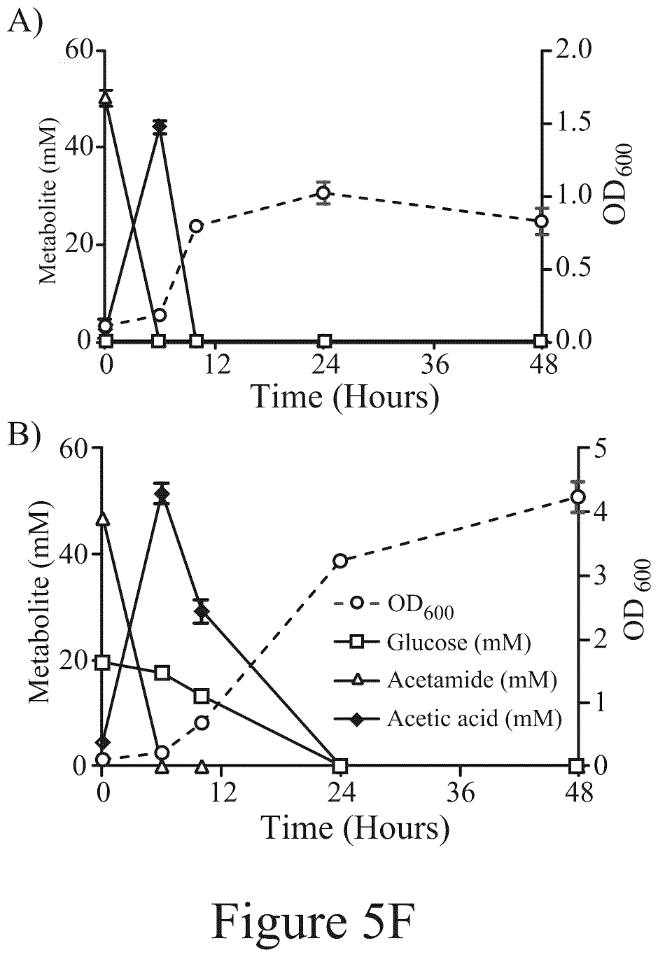

While these plasmid overexpression data looked promising, their applicability in an industrial sense is potentially limited due to the need for constant antibiotic selection pressure. Therefore, genomically integrated strains were generated to test amidase functionality in a more industrially relevant context without the need for antibiotics. The strong constitutive promoter P_{tac} was placed in front of PP_0613 (strain RE026), which is a member of a two gene operon with PP_0614 being upstream. Expression of the operon was insulated with the addition of a terminator sequence downstream of the PP_0614 stop codon and upstream of the Pt_{ac} sequence allowing expression of PP_0613 only. A second strain, RE020, was generated where the coding sequence of PP_0613 was replaced with the amiE gene again under the control of an insulated P_{tac} promoter (see FIGS. 5A and 5D, respectively).

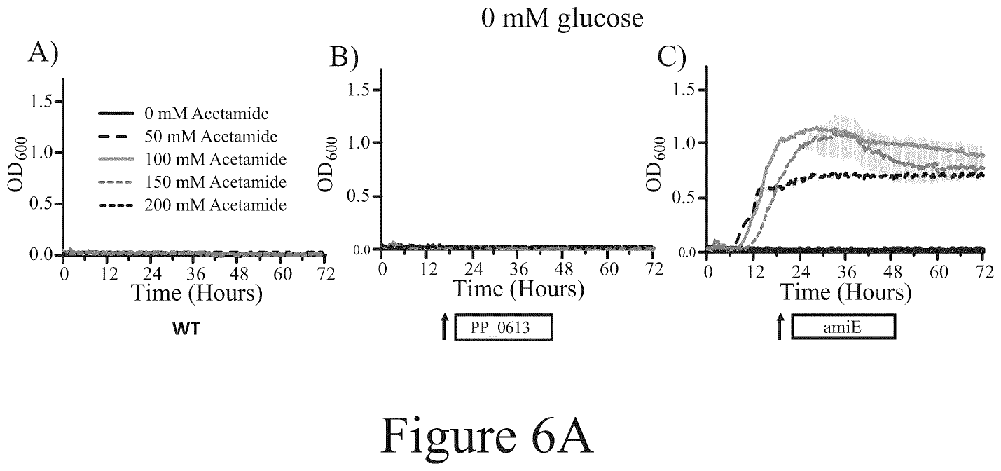

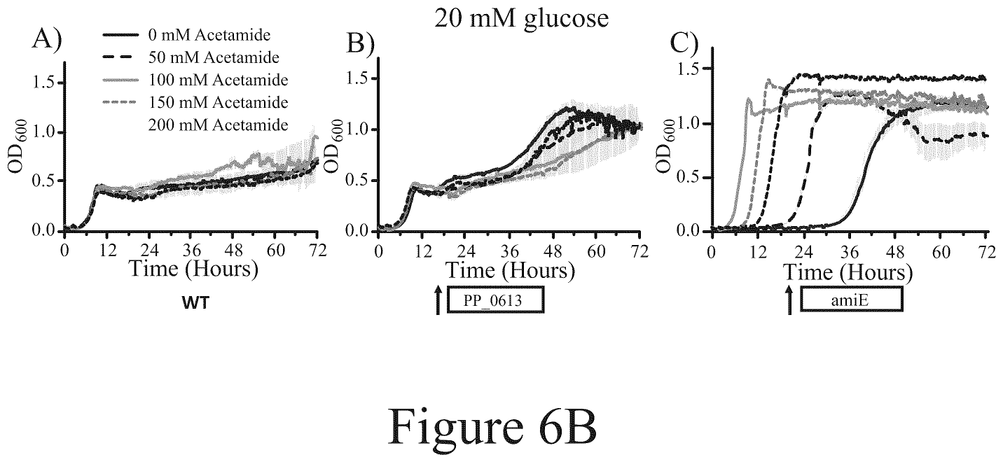

The growth of both RE020 and RE026 was tested in M9 minimal media containing acetamide as the sole carbon source. Interestingly, RE026 (overexpressing PP_0613) was unable to grow on acetamide at any of the given concentrations while RE020 grew on acctamide up to 150 mM (see FIGS. 6A and 6B). Differences in gene copy number when expressed from a plasmid as opposed to the genome may have led to this discrepancy.

Next, the ability of these engineered strains to grow in minimal media conditions was assessed where acetamide was providing all the necessary nitrogen, as well as carbon, for growth. RE026 again was unable to grow or utilize acetamide in any of the conditions tested, as expected given previous results. Albeit some growth was seen in microtiter plate experiments when cells were grown with 20 mM glucose supplement. Shake-flask experiments likewise showed a very slow breakdown of acetamide by RE026 only when glucose was present, resulting in very slow growth, likely due to the lack of uscable nitrogen in the media (see FIGS. 5B and 5C). RE020 on the other hand was able to grow in microtiter plate experiments with M9 minimal media lacking nitrogen with acetamide providing the only carbon and nitrogen source for growth (see FIGS. 5E and 5F). At 200 mM acetamide the cells had a much longer lag-time and did not reach the same OD₆₀₀ levels as other conditions, possibly due to toxicity effects from the production of high concentrations of ammonium. In shake-flask experiments, 50 mM acetamide was converted to acetate within 10 hours and cell growth was observed both in the absence and presence of exogenous glucose (see FIG. 5F). These data together suggest that chromosomal expression of amiE but not PP_0613 can facilitate the breakdown of acetamide and growth of engineered P. putida in minimal media containing no additional nitrogen.

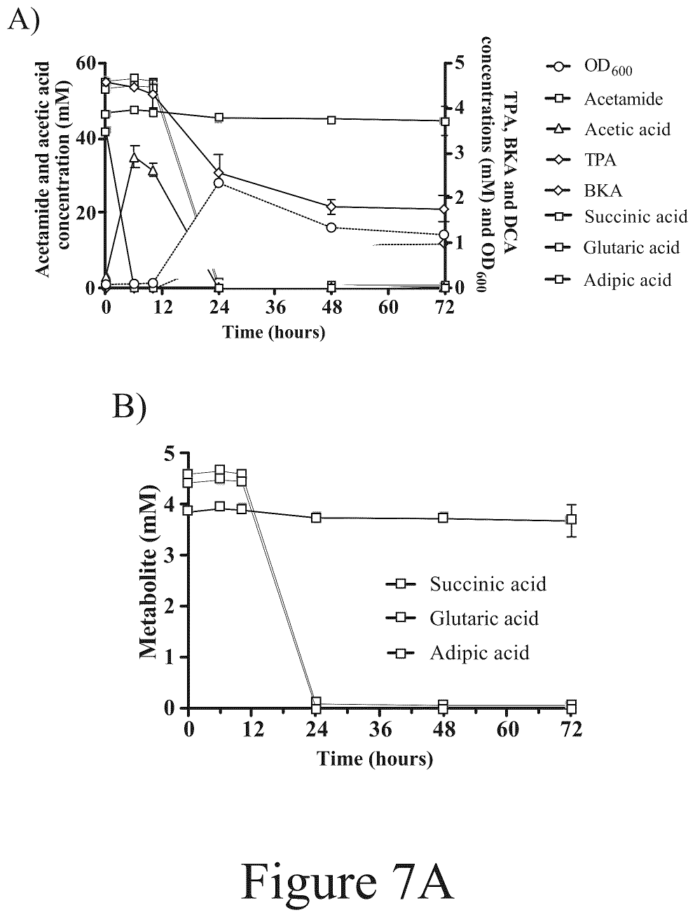

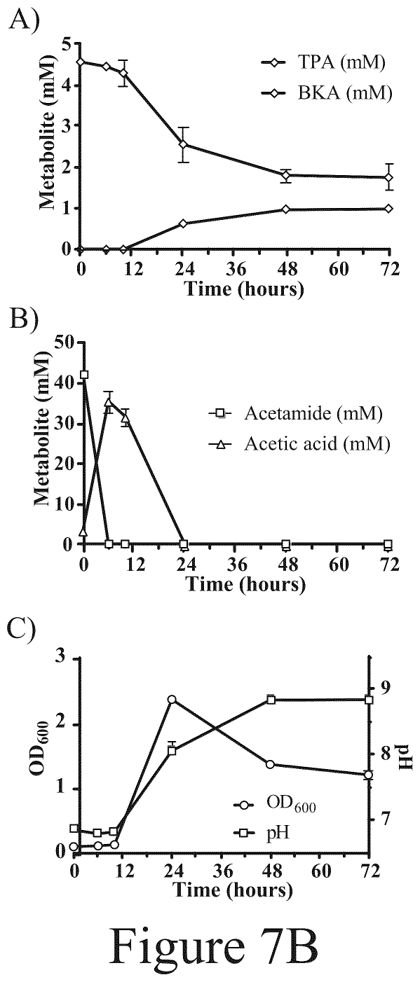

Oxidation of nylon-6 can result in a mixture of short chain DCAs as well as acetamide. Under these same conditions, PET can be oxidized into TPA, which can be biologically converted into valuable products such as muconolactone and beta-ketoadipate (BKA). For these reasons, a strain of P. putida was engineered that could utilize DCAs and acetamide as carbon and nitrogen sources for growth while funneling TPA into one of these products. Strain AW307 has been previously shown to utilize DCAs and can funnel TPA to BKA. To enable utilization of acetamide AW307 and amiE were integrated under the control of P_{tac} in the PP_0613 locus resulting in strain RE022. In an initial shake-flask experiment, RE022 was grown in the presence of 50 mM acetamide, 5 mM of each C4-C6 DCA, and 5 mM TPA in M9 media with no supplementary nitrogen. Acetamide was completely utilized by 6 hours with the resulting acetate being consumed by 24 hours. Only around 40 mol % of the supplied TPA was consumed with around 22 mol % converted into BKA (see FIGS. 7A and 7B).

Cell growth was also inhibited after 24 hours. Subsequent analysis showed that there was an increase in pH to ˜9 likely due to the large increase in non-utilized ammonium which has a pKa of 9.25 and likely overwhelmed the buffering capacity of the media. It was also apparent that RE022 was unable to utilize adipic acid as a carbon source (see Panel A of FIG. 7A and Panel C of FIG. 7B). This observation makes sense given that AW307 was also unable to utilize this adipic acid. To accumulate BKA, RE022 (and AW307) contains a knockout of pcaIJ, a 3-oxoadipate-CoA transferase operon. It seems likely that removal of this operon resulted in an inability of cells to add CoA to adipic acid for breakdown to succinic acid. This was seen previously where AW162, which does not contain a pcaIJ knockout, can fully utilize adipic acid. With this in mind, a redesign of the production pathway from TPA to muconolactone was undertaken.

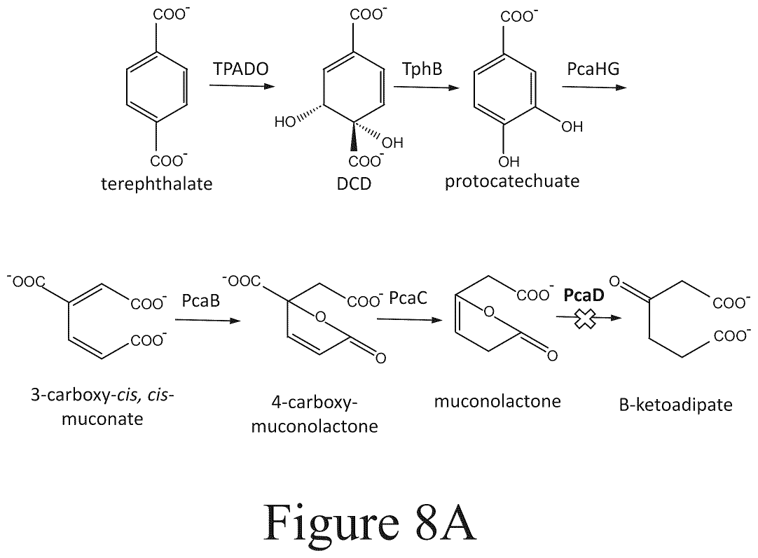

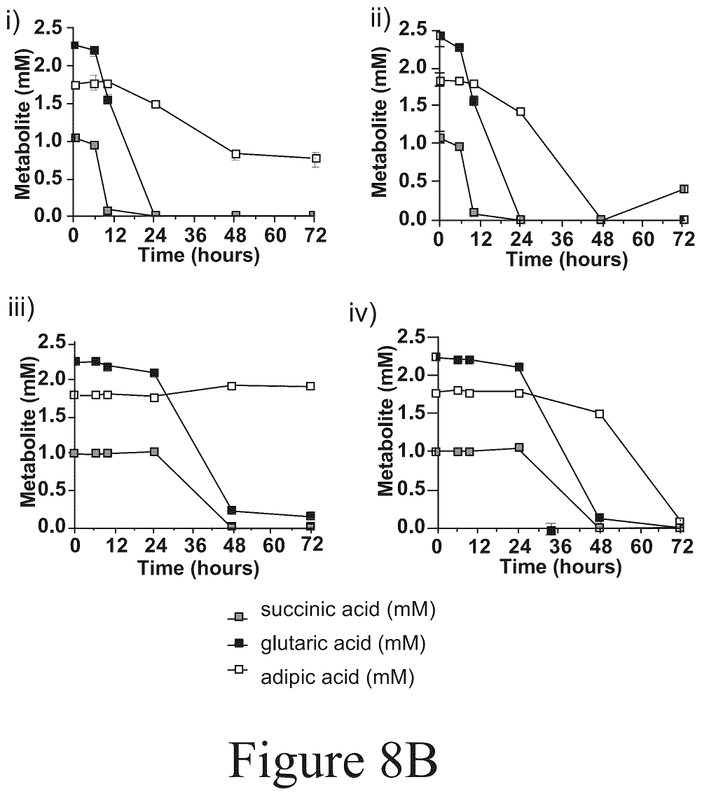

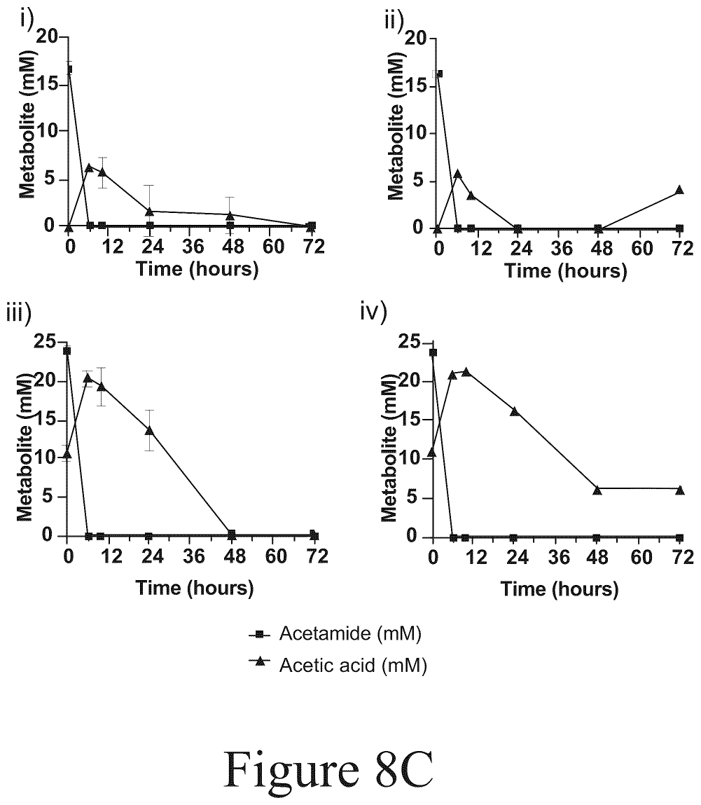

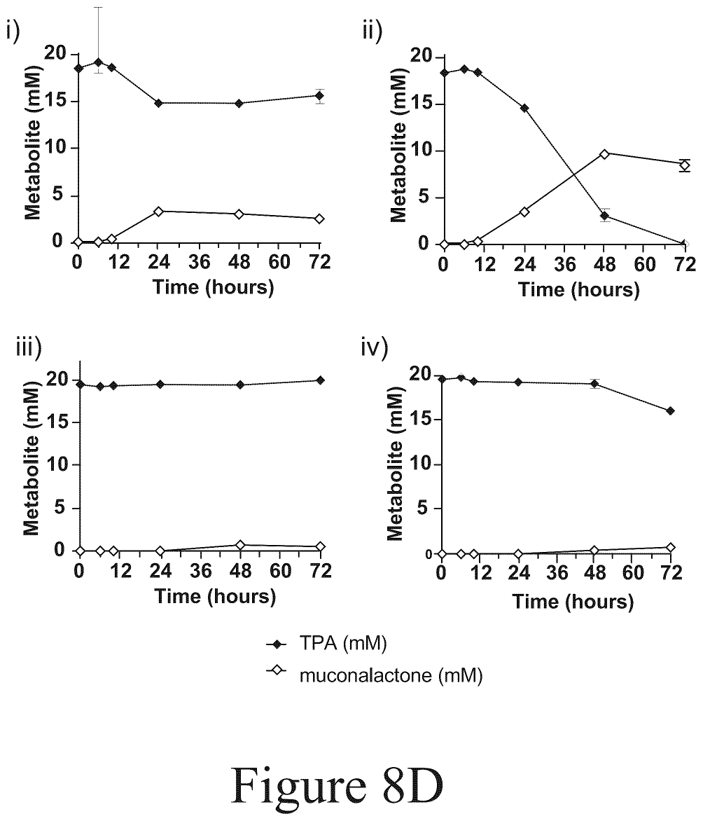

Muconolactone is one step above BKA in the aromatic degradation pathway of P. putida, with conversion of muconolactone to BKA being facilitated by the 3-oxoadipate enol-lactonasc PcaD (sec FIG. 8A). Knocking out the pcaD gene therefore should truncate the TPA degradation pathway at muconolactone allowing accumulation of this valuable product. To this end, previously engineered strain AW162 was chosen, known to consume adipic acid and shorter chain DCAs, and P_{tac} amiE was inserted for amidase overexpression, and pcaD knocked out for funneling of TPA to muconolactone, resulting in strain RE093. To better mimic the conditions of actual oxidative deconstruction of PET and nylon-6, the oxidation was performed, the effluent prepared for bio-funneling, and the resulting DCA, acetamide, and TPA mixture quantified. The results of this quantification were then used to perform a mock experiment in parallel to the experiment utilizing actual effluent. Given that the cells may be carbon limited due to the relatively low concentrations of acetamide and DCAs, it was decided to run a fed-batch experiment in parallel to try and achieve the highest possible conversion of TPA to muconolactone. In both batch and fed-batch modes, acetamide was fully utilized and converted into acetate within 6 hours, as observed in previous experiments, showing the activity of AmiE in this strain. Likewise, DCAs were fully utilized in both modes within 48 hours suggesting that PcaIJ is indeed involved in the metabolism of adipic acid despite the presence of the non-native DcaIJ from Acinetobacter baylyi ADP1.

Oxidation reactions were run on mixed PET and nylon-6 model materials to confirm that in such a mixed setting the resulting depolymerization products matched those seen before. Indeed, at the previously determined PET depolymerization conditions both PET and nylon-6 were broken down into TPA (from PET) and DCAs and acetamide (from nylon-6). Following evaporation of acetic acid, this depolymerized waste material was resuspended in H₂O and treated to remove residual metal catalysts from the effluent. The resulting solution was neutralized to pH ˜7 before being used to prepare M9 minimal media for bacterial cultivation.

Next, the efficacy of this tandem approach was assessed when using post-consumer textile waste products containing nylon-6 and PET. To achieve this, a 1:1 mixture of a 100% PET garment and a 100% nylon-6 garment was used. The effluent was prepared in the same manner as previously and analytical results again indicated the presence of the expected oxidation products. Therefore, this post-consumer waste effluent was supplied to the engineered bacteria and again tracked substrate utilization and bioconversion of TPA to muconolactone.

### Experimental

Reaction conditions for polymer oxidation: A titanium PARR reactor with a 1″ Teflon coated stir bar and a glass insert were charged with 20 mL of glacial acetic acid, 9.7 wt % cobalt (II) acetate, 9.5 wt % manganese (II) acetate, 6.7 wt % zirconium (IV) acetylacetonate, 22.5 wt % N-hydroxyphthalimide (NHPI, where used), and 350 mg of each polymer substrate (where used). The reactors were then sealed and purged three times with ultra-high purity (UHP) nitrogen before pressure testing at 90 bar. Given passing the leak test, the reactors were charged with ultra-zero compressed air (typically 38 bar) and diluted with UHP nitrogen (typically to 80 bar total pressure). The reactors were then heated at the desired temperature and reaction time. Following, the reactions are cooled on ice to room temperature before being depressurized and filtered for analysis. All reaction mixtures were filtered through a 0.2 μm frit. Nylon products were diluted with acetone, tetrahydrofuran (THF), or dimethylsulfoxide (DMSO) for LC-MS analysis. Solids produced from PET were recovered and dried overnight in a vacuum oven at 40° C., along with the filtrate, and diluted with THF.

Bacterial strains and cultivation conditions: All bacterial cultivations for plasmid and strain construction were grown in LB media supplemented with 50 μg/mL kanamycin as required. E. coli were grown at 37° C. shaking at 225 rpm while Pseudomonas putida were grown at 30° C. shaking at 225 rpm. Glycerol stocks were prepared in 20% (w/v) glycerol and stored at −80° C. Following strain confirmation Pseudomonas putida were grown in M9 minimal media with nitrogen (6.78 g/L K₂HPO₄, 3 g/L KH₂PO₄, 0.5 g/L NaCl, 1 g/L NH₄C₁, 2 mM MgSO₄, 100 μM CaCl₂, and 18 μM FeSO₄) or without nitrogen (6.78 g/L K₂HPO₄, 3 g/L KH₂PO₄, 0.5 g/L NaCl, 2 mM MgSO₄, 100 μM CaCl₂, and 18 μM FeSO₄), containing supplementary carbon as indicated in each experiment, and grown at 30° C. with shaking at 225 rpm.

Plasmid construction: Plasmid construction details are listed below (SEQ ID NOs: 5 and 6). plasmid DNA constructs and gBlocks were synthesized by Twist Biosciences, and single stranded oligonucleotide primers were synthesized by Integrated DNA technologies. DNA was amplified by polymerase chain reaction (PCR) using Q5® High-Fidelity 2X master mix for cloning reactions and MyTaq™ Red Mix for colony PCR reactions. Plasmid DNA was assembled using the NEBR NEBuilder HiFi DNA assembly master mix using 20 bp overhangs. 3 μL of assembly reactions was transformed directly into NEB® 5-alpha F′I^{q} chemically competent E. coli. Plasmid sequences were confirmed by whole plasmid nanopore sequencing (Plasmidsaurus).

Strain construction: Strain construction and genotype details are listed below (SEQ ID NOs: 7 and 8). Chromosomal deletions and insertions were performed in Pseudomonas putida KT2440 using the antibiotic selection/sacB counterselection method as previously described. In brief, plasmid DNA was introduced to cells via electroporation and transformed cells were plated on LB plates containing 50 μg/mL kanamycin. Resulting colonies were re-streaked onto fresh LB+kanamycin plates before individual colonies were plated on YT agar plates containing 25% (w/v) sucrose for counterselection. Individual colonies were re-streaked onto fresh YT+sucrose plates before backbone removal and complete recombination were confirmed by both colony PCR and susceptibility to kanamycin. Integrations and knockouts were confirmed by cPCR and nanopore sequencing as well as whole genome sequencing of all final strains using nanoporc sequencing (Plasmidsaurus).

Microtiter plate cultivations: Pseudomonas putida strains were streaked out onto LB plates (with antibiotics as required) from glycerol stocks. Single colonies were used to inoculate 5 mL of LB (with antibiotics as required) and grown overnight at 30° C. at 225 rpm. Cells were pelleted at 4,000 g and washed once with 1X M9 salts (with or without NH₄Cl as required) and inoculated into fresh M9 media in triplicate with supplemental carbon and nitrogen as indicated in each experiment. OD₆₀₀ readings were measured in a BioscreenC® (Growth Curves, USA) from 200 μL cultivations grown in Honeycomb 100-well plates at 30° C. with maximum orbital shaking and a measurement interval of 15 minutes.

Shake-flask cultivations: See microtiter plate cultivations for details on preculture preparation. Washed cells were inoculated at an OD₆₀₀ of 0.1 into 30 mL of fresh M9 media in 125 mL Erlenmeyer flasks in triplicate. Carbon and nitrogen sources were supplied as described for each individual experiment. All cultivations were performed at 30° C. with shaking at 225 rpm. For metabolite analysis 1 mL of culture was taken at the desired timepoint, spun at >18,000 g for 2 minutes and filtered into HPLC vials using a 0.2 μm syringe filter. Samples were diluted as necessary for analytical procedures and stored at −20° C. prior to analysis.

The embodiments described herein should not necessarily be construed as limited to addressing any of the particular problems or deficiencies discussed herein. References in the specification to “one embodiment”, “an embodiment”, “an example embodiment”, “some embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, ±10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

The foregoing discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

What is claimed is:

1. An engineered microorganism comprising an exogenous gene encoding an amidase, wherein the microorganism is capable of catabolizing acetamide to produce a carboxylic acid without the use of an additional substrate.

2. The engineered microorganism of claim 1, wherein the additional substrate comprises a sugar, an alcohol, a protein, or a nucleic acid.

3. The engineered microorganism of claim 1, wherein the carboxylic acid is acetate.

4. The engineered microorganism of claim 1, wherein the engineered microorganism comprises a species of Pseudomonas.

5. The engineered microorganism of claim 4, wherein the species comprises a strain of Pseudomonas putida.

6. The engineered microorganism of claim 5, wherein of the strain comprises KT2440.

7. The engineered microorganism of claim 4, wherein the exogenous gene is derived from a species of Pseudomonas that is not Pseudomonas putida.

8. The engineered microorganism of claim 7, wherein the exogenous gene is derived from Pseudomonas aeruginosa.

9. The engineered microorganism of claim 8, wherein the exogenous gene comprises amiE.

10. The engineered microorganism of claim 9, wherein the exogeneous gene has a nucleic acid sequence that is at least 80% identical to SEQ ID NO:1.

11. The engineered microorganism of claim 9, wherein the amidase has an amino acid sequence that is at least 80% identical to SEQ ID NO:2.

12. The engineered microorganism of claim 1, wherein the exogenous gene is chromosomally incorporated into the engineered microorganism.

13. The engineered microorganism of claim 1, further comprising the deletion of an endogenous gene encoding an amidase.

14. The engineered microorganism of claim 13, wherein the endogenous gene has a nucleic acid sequence that is at least 80% identical to SEQ ID NO:3.

15. The engineered microorganism of claim 13, wherein the endogenous gene encodes an amino acid sequence that is at least 80% identical to SEQ ID NO:4.

16. A method comprising:
depolymerizing a polyamide to produce a mixture comprising acetamide, and
using an engineered microorganism, converting at least a portion of the acetamide to a carboxylic acid, wherein:
the engineered microorganism comprises an exogenous gene encoding an amidase, and
the microorganism is capable of catabolizing acetamide to produce a carboxylic acid without the use of an additional substrate.
